(19) United States
(12) Patent Application Publication
(10) Pub. No.: US 2019/0223931 A1
(43) Pub. Date: Jul. 25, 2019

(54) MIXING AND DISCHARGE SYSTEM FOR BONE REPLACEMENT MATERIAL

(71) Applicant: MEDMIX SYSTEMS AG, Rotkreuz (CH)

(72) Inventor: Benjamin NIEBER, Eschenbach (CH)

(73) Assignee: MEDMIX SYSTEMS AG, Rotkreuz (CH)

(21) Appl. No.: 16/311,825

(22) PCT Filed: Jun. 19, 2017
(86) PCT No.: PCT/EP2017/064947

§ 371 Date: Dec. 20, 2018

(30) Foreign Application Priority Data
Jul. 12, 2016 (CH) 00889/16

(51) Int. Cl. A61B 17/88; A61F 2/46

(52) U.S. Cl. CPC A61B 17/8825; A61B 2017/8838; A61B 17/8833; A61F 2/4644

(57) ABSTRACT

A mixing and delivery system (1) and a method for impregnating granules (G) with a liquid and for delivering a resulting mixture are specified. The mixing and delivery system (1) has a container (2) for holding the granules. In a distal end region (25) of the container is arranged a separation element (4), which is designed to retain granules but allow liquid to pass through. The container has a predefined breaking point (27) proximal of the separation element (4) so that the distal end region between the predefined breaking point and the distal container end (23) can be broken off from the container together with the separation element (4), thereby creating a delivery opening on the container for delivering the mixture.

### TECHNICAL FIELD

The present invention relates to a mixing and discharge system and a corresponding method for impregnating granulate with a liquid and for discharging the mixture.

### PRIOR ART

Bone graft materials find an application in various fields of medicine, such as dentistry, orthopedics or reconstructive surgery. The bone graft material consists mostly of a granular (particulate) phase of natural or synthetic origin (for example a two-phase calcium phosphate/hydroxyl apatite material) and a liquid phase (for example endogenous blood or a physiological sodium chloride solution). The bone graft material usually has a paste-like consistency.

The granular material, hereinafter referred to as granulate, is traditionally supplied in a bowl and the liquid phase is added, whereby the granulate is wetted. In order to apply the resultant finished bone graft material to the patient, it is brought to the intended destination with a spatula. This may prove to be difficult, or even impossible, depending on the location of the intended destination, and, since the material lies in a loose pile on the spatula, there is a risk of the material falling from the spatula in the course of its discharge. An operation of this kind is time-consuming in addition, and care must be taken during preparation to ensure that the granulate and the liquid phase are mixed together in the correct proportion, so that the bone graft material has the desired paste-like consistency.

It has therefore been proposed in the prior art to take up the granulate in a syringe and to wet it with the liquid phase directly in the syringe. In order to retain the granulate in the syringe, but to facilitate the admission of the liquid phase into the syringe, it is known to fit a filter or a sieve to the syringe.

A granulate syringe, in which a cap with a disk made of a porous material is pushed or screwed onto the outlet end of the syringe housing, is known from EP 0 470 393 B1. In order to deliver the mixture, the cap is removed and a piston is pushed forwards.

EP 1 093 767 B1 discloses a syringe having a syringe cylinder, which is filled with granulate. A needle tip, which possesses a sieve in a recess, is attached to the cylinder by means of a friction fit. Blood is sucked into the syringe cylinder via an opening. If the blood is mixed sufficiently with the granulate, the needle tip is pushed downwards manually from the syringe cylinder.

A syringe for the application of bone graft material, which has a cylinder in which granulate is taken up, is disclosed in EP 2 436 342 B1. The cylinder has at its distal end an external thread, on which a removable attachment with an internal thread is held. The attachment has an injection opening as well as drainage openings, through which liquid can be injected or sucked into the cylinder. If the bone graft material is sufficiently impregnated with liquid, the attachment is screwed off and replaced by a curved discharge nozzle.

In suchlike syringes, however, there is still room for improvement with regard to their operation.

### SUMMARY OF THE INVENTION

An object of the present invention is to provide a mixing and discharge system for impregnating granulate with a liquid and for discharging the mixture, which ensures the effective impregnation of the granulate and discharge of the mixture and offers simple operation in the process. The mixing and discharge system should also lend itself to simple and cost-effective manufacture.

This object is accomplished by a mixing and discharge system having the features of claim 1. Further embodiments are proposed in the dependent claims.

A mixing and discharge system for the impregnation of granulate with a liquid and for the discharge of a mixture resulting therefrom is thus proposed, which has a syringe-like container for holding the granulate having a circumferential container wall, a proximal container end and a distal container end. The container can constitute in particular an elongated cavity having an essentially constant cross section. The distal container end forms a container opening for introducing the liquid into the container. A feed element displaceably mounted in the container serves for discharging the mixture in a distal discharge direction. A separation element, which is designed to retain granulate, but to allow liquid to pass through, is arranged in a distal end region of the container. The container has a predefined breaking point, proximal to the separation element, so that the distal end region situated between the predefined breaking point and the distal container end can be broken off from the container together with the separation element, thereby creating a discharge opening on the container for discharging the mixture.

On the one hand, the granulate is able to be retained in the container and impregnated effectively with liquid thanks to the presence of the separation element. A sieve with a plurality of defined sieve openings or a filter, for example, can serve as a separation element. In particular, the separation element can be designed as a flat plastic disk produced by the injection molding process having a multiplicity of defined passage openings. On the other hand, once impregnation of the granulate with liquid is complete, the separation element can be removed in a very simple manner because of the predefined breaking point. The resulting mixture can be discharged directly through the discharge opening that has been created in this way, without the need to fit an attachment or the like to the mixing and discharge system. The operation is facilitated as a result and is very intuitive.

According to a first embodiment, the separation element can be configured in one piece with the container wall. A separation element that is configured in one piece or integrally with the container wall exhibits the advantage that production of the container together with the separation element can be performed in the same production step. This can take place in particular in the injection molding process.

According to further embodiments, however, the separation element can also be configured separately from the container. This has the advantage that the separation element can be adapted very easily to the requirements of various kinds of granulate and the liquid phase, for example to various particle sizes of the granulate, but without the need to change the injection mold for the entire container. A single kind of container can be produced, which can then be provided with the appropriate separation element depending on the requirements. At the same time, the separation element can be produced from the same material or a different material as the container, depending on the requirements.

The separation element is secured advantageously inside the container, at least against displacement in the distal discharge direction, and preferably also in the opposing (proximal) direction. For this purpose, the container wall can have at least one appropriate retaining means (for example in the form of at least one inwardly projecting protrusion in the form of a nose or a circumferential bead, or in the form of an internal groove), which secures the separation element in the container against displacements at least in the distal direction, and preferably also in the proximal direction.

In addition, the separation element can be secured against rotations about the longitudinal axis of the container by the same or a different retaining means, which engages along the circumference of the separation element. This is of advantage in particular when the separation element is connected initially to a closure, as described in more detail below.

The mixing and discharge system advantageously further comprises a removable closure in order to close the container opening. The provision of a closure excludes the possibility of contamination of the container opening and the contents of the container as a result of environmental influences prior to use of the mixing and discharge system. The closure is removed from the container to permit the use of the mixing and discharge system, so that the liquid can be admitted into the container through the container opening. After impregnation of the granulate with the liquid has taken place, the closure can be replaced on the container, if necessary, in order to close the container opening once more. The closure in this case can serve as an aid to breaking to permit easier breaking-off of the distal end region.

It is preferable for the closure to comprise a plug, which extends against the discharge direction into the distal end of the container through the container opening as far as the separation element. A suchlike closure prevents fine granulate particles (“dust”), which is often unavoidably present in the granulate, from passing through the openings of the separation element and accumulating between the separation element and the closure.

The closure may be capable of being attached to the container, for example by means of a simple plug-in connection. As an alternative, it may be capable of being attached to the container by means of a threaded connection. Different embodiments of a threaded connection are conceivable in this respect. For example, the closure may have an internal thread, which enters into engagement with a corresponding external thread on a distal external side of the container wall if the closure is screwed or pressed axially onto the container. An external thread on the plug is conceivable, on the other hand, which enters into engagement with a corresponding internal thread on a distal internal side of the container wall if the plug is screwed or pressed axially into the container opening. There is a particular preference for a round thread or a buttress thread to be provided, so that the closure can be pressed lightly onto the container opening or pressed into the container opening, against the discharge direction, although it can only be removed from the discharge opening by means of a screwing movement in the discharge direction.

In advantageous embodiments, the closure is connected initially to the separation element, wherein the separation element is attached to the container in such a way that the separation element remains inside the container if the closure is removed from the container. This has the advantage that the separation element can be introduced very easily into the container in conjunction with assembling the mixing and discharge system. The separation element thus arrives automatically at the designated location in the distal end of the container when the closure is positioned on the container.

The closure in this case is preferably embodied in one piece with the separation element and, in particular, is connected to the separation element by means of a predefined breaking point. The unit comprising the closure and the separation element can in turn be produced by the injection molding process.

It is of particular advantage in this case for the closure to be attached to the container by means of a threaded connection, in particular having a round thread or a buttress thread, as described above. In this case, the closure together with the separation element can be connected to the container by being pressed in or pressed on axially in conjunction with assembling the mixing and discharge system. The separation element preferably engages in the retaining means of the container wall in this case and is secured inside the container as a result thereof. The closure can then be removed from the container once more without tools by means of only a screwed connection. As a result, the connection between the closure and the separation element ruptures without the need for the application of excessive force by the user. The separation of the closure from the separation element then takes place particularly easily if the separation element is secured not only axially, but also in relation to rotations in the container. In this case, not only tensile forces, but also torsional forces, act upon the connection between the closure and the separation element in conjunction with unscrewing the closure, which facilitates the separation of the connection.

The container preferably has a curvature and forms, distally from the curvature, an angled section which extends outwards from the central longitudinal axis in relation to a central longitudinal axis of the container, wherein the angled section extends outwards away from the central longitudinal axis preferably at an angle of curvature of between 200 and 500, and wherein the distal end region is arranged in the angled section.

In other words, both the separation element and the predefined breaking point are thus present in the angled section of the container, so that the discharge opening, once the distal end region has been broken off, is also present in the angled section of the container. A suchlike angled embodiment of the container enables the user to perform precisely targeted discharge of the mixture at the point of application.

In order to make easier breaking off of the distal end region of the container available to the user, breaking aids may be provided at the distal end region. For example, longitudinal ribs, retaining wings or transverse grooves, which are grasped by the user for the breaking-off operation and which ensure a better grip, can be provided externally in the distal end region.

The feed element advantageously has a piston, which is connected to a piston rod. In order to reduce the friction between the piston rod and the internal wall of the container, it is advantageous for the piston rod to have an external diameter which is smaller than the internal diameter of the container. In order nevertheless to ensure good guidance of the piston rod, ring-shaped guide beads may be formed on the circumferential surface of the piston rod. If the container exhibits a curvature, it is also advantageous for the piston rod to exhibit a region of reduced external diameter in a distal region, wherein the reduced diameter is smaller than the external diameter in the proximal region. In this way, the piston rod is more flexible and is able to follow the curvature more easily. Guide beads may also be positioned on the piston rod in the region of reduced external diameter, so that good guidance of the piston rod in the container is still ensured.

The container can be pre-filled with granulate having a medium-sized granulate diameter. The separation element then preferably has passage openings, of which the diameter is smaller than the medium-sized granulate diameter.

The mixing and discharge system described above can be used as follows. The mixing and discharge system is provided initially with granulate, and the granulate is impregnated with a liquid. In order to create the discharge opening for discharging the mixture on the container, the distal end region together with the separation element is broken off at the predefined breaking point. In order to discharge the mixture from the discharge opening, the piston rod is displaced in the distal discharge direction.

Impregnation of the granulate preferably takes place by sucking up the liquid by causing the feed element to move against the discharge direction. For example, the discharge opening can be immersed in a reservoir of liquid for this purpose.

As an alternative, impregnation of the granulate can take place by injection of the liquid through the container opening and through the separation element. For example, a moistening syringe can be filled with liquid for this purpose, wherein the syringe needle is passed through the separation element and the liquid is thereby fed to the granulate. It is of advantage in this respect for the needle to be introduced into the container through a preformed opening in the separation element.

### BRIEF DESCRIPTION OF THE DRAWINGS

Preferred embodiments of the invention are described below with the aid of the drawings, which serve only to provide an explanation and must not be interpreted in a restrictive way. In the drawings:

FIG. 1 depicts a perspective view of a mixing and discharge apparatus having a closure according to a first embodiment;

FIG. 2 depicts a central longitudinal section through the mixing and discharge apparatus in FIG. 1;

FIG. 3 depicts a detailed view of region A in FIG. 2 having a separation element according to a first embodiment;

FIG. 4 depicts a detailed view of region A in FIG. 2 having a separation element according to a second embodiment;

FIG. 5 depicts a detailed view of region A in FIG. 2 having the separation element of the first embodiment, but having a closure according to a second embodiment;

FIG. 6 depicts a perspective view of the mixing and discharge apparatus in FIG. 1 having a closure according to a third embodiment;

FIG. 7 depicts a central longitudinal section through the mixing and discharge apparatus in FIG. 6;

FIG. 8 depicts a detailed view of region B in FIG. 7;

FIG. 9 depicts a central longitudinal section through a mixing and discharge apparatus having a closure according to a fourth embodiment, wherein the closure constitutes a unit having a separation element according to a third embodiment;

FIG. 10 depicts a detailed view of region C in FIG. 9;

FIG. 11 depicts a detailed view of region C in FIG. 9 having a closure according to a fifth embodiment, wherein the closure constitutes a unit having a separation element according to a fourth embodiment;

FIG. 12 depicts a detailed view of region C in FIG. 9 having a closure according to the fifth embodiment and a separation element according to a fifth embodiment;

FIG. 13 depicts a detailed view of region C in FIG. 9 having a closure according to a sixth embodiment and a separation element according to the fourth embodiment;

FIG. 14 depicts a detailed view of region C in FIG. 9 having a closure according to the sixth embodiment and a separation element according to the fifth embodiment;

FIG. 15 depicts a detailed view of region D in FIG. 13;

FIG. 16 depicts a detailed view of region E in FIG. 14;

FIG. 17 depicts a central longitudinal section through the mixing and discharge apparatus in FIG. 9, after the closure has been removed;

FIG. 18 depicts a detailed view of region H in FIG. 17;

FIG. 19 depicts an illustration of a filling process according to one variant;

FIG. 20 depicts an illustration of a filling process according to a second variant;

FIG. 21 depicts a detailed view of region G in FIG. 20;

FIG. 22 depicts a central longitudinal section through a mixing and discharge apparatus having a broken-off end region;

FIG. 23 depicts a detailed view of region H in FIG. 22;

FIG. 24 depicts a perspective view of a container of the mixing and discharge apparatus having an aid to breaking according to a first embodiment;

FIG. 25 depicts a perspective view of a container of the mixing and discharge apparatus having an aid to breaking according to a second embodiment;

FIG. 26 depicts a perspective view of a container of the mixing and discharge apparatus having an aid to breaking according to a third embodiment;

FIG. 27 depicts a central longitudinal section through a mixing and discharge apparatus after removal of the end region in an initial state at the start of the discharge process;

FIG. 28 depicts the mixing and discharge apparatus in FIG. 27 in an intermediate state, after a part of its contents has been discharged; and

FIG. 29 depicts the mixing and discharge apparatus in FIG. 24 in a final state, after the whole of its contents has been discharged.

### DESCRIPTION OF PREFERRED EMBODIMENTS

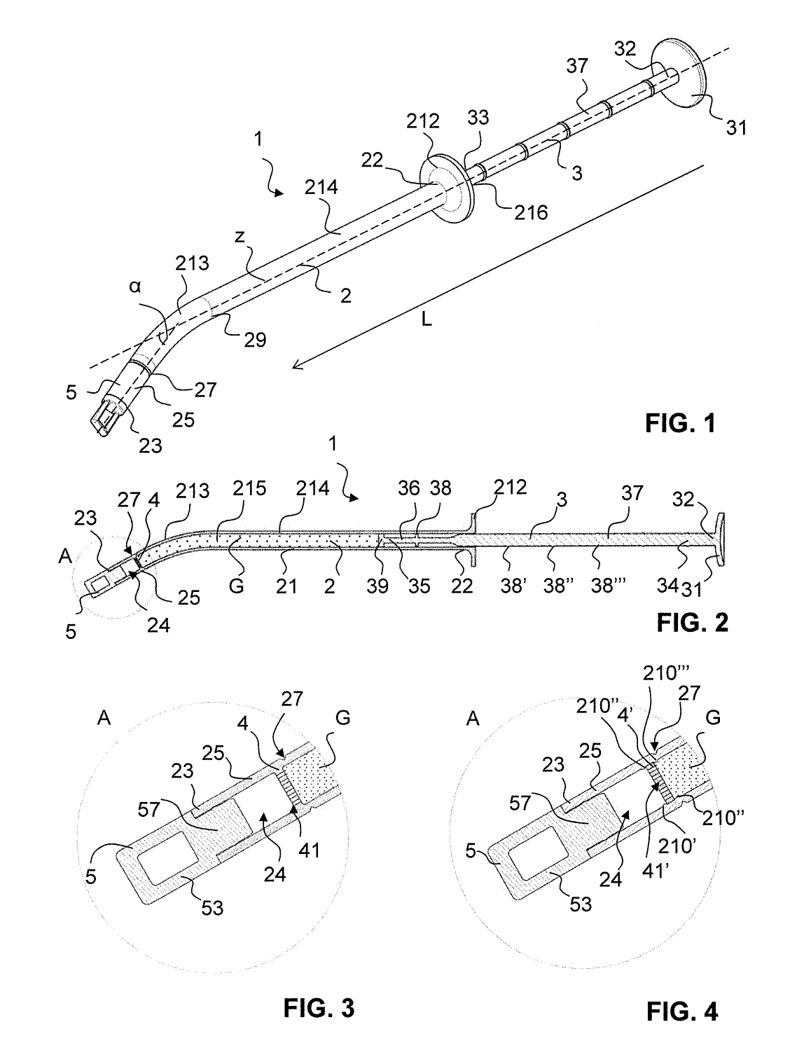

In FIGS. 1 to 3, a first embodiment of an inventive mixing and discharge apparatus 1 is illustrated in various views. The mixing and discharge apparatus 1 comprises a container 2 having a separation element 4 as well as a feed element 3 displaceably mounted in the container 2 for discharging a mixture in a distal discharge direction L. The container 2 is closed by a closure 5.

The distal discharge direction L is defined as the direction along which the feed element 3 moves into the container 2, in order to discharge the mixture from the container 2. The direction opposite thereto is referred to as the proximal direction.

The container 2 has a circumferential container wall 21, which delimits a cylindrical interior space 215 of the container for receiving a granulate G, as well as a proximal container end 22 and a distal container end 23.

The proximal container end 22 forms an inlet opening 216, which opens into the interior space 215 of the container. Once the granulate G has been filled into the container 2 via this inlet opening 216, the feed element 3 is also introduced into the container 2 through the inlet opening 216. At the proximal end 22, the container 2 has a retaining plate 212 surrounding the central longitudinal axis Z. Retaining wings can also be provided as an alternative, of course, as is often the case in conjunction with syringes.

The distal container end 23 forms a container opening 24 for admitting a liquid F into the interior space 215 of the container. As can be appreciated from FIG. 3 in particular, the separation element 4 is arranged in the form of a filter or a sieve in a distal end region 25 of the container 2. It serves to retain the granulate G in the interior space 215 of the container, but to allow the penetration of liquids F into the interior space 215 of the container.

The feed element 3 has a piston rod 37, at the distal end 33 of which a piston 39 is formed. The piston rod 37 has at its proximal end 32 a pressure plate 31, configured as a thumb rest, which is configured for the purpose of causing the piston rod 37 to advance in the distal discharge direction L in order to discharge the mixture, or to draw it back against this discharge direction in order to admit liquid into the container 2.

As explained in detail below in conjunction with FIGS. 22 to 26, the container 2 has a predefined breaking point 27 proximally to the separation element 4, so that the distal end region 25 of the container 2 situated between the predefined breaking point 27 and the distal container end 23 can be broken off from the container 2 together with the separation element 4, wherein a discharge opening 28 is formed for discharging the mixture to the container 2.

As shown in more detail in FIGS. 1 and 2, the container 2 has a region of curvature 29 and forms an angled section 213 distally to the curvature. The angled section 213 extends outwards from the central longitudinal axis Z at an angle of curvature a of about 30° in relation to a central longitudinal axis Z of the container 2. Proximally to the curvature, the container 2 has a straight region 214, which extends parallel to the central longitudinal axis Z of the container 2.

As can be seen in particular in FIG. 2, the separation element 4 is arranged distally to the straight region 214 in the angled section 213 of the container 2. The separation element 4 fills the clear cross-section of the interior space 215 of the container completely and is produced in one piece with the container wall 21.

In order for liquids to be capable of being admitted into the interior space 215 of the container, the separation element 4 has passage openings 41, which extend completely through the separation element 4 in the discharge direction L. In order, on the one hand, for liquids to be capable of being admitted into the container 2, but, on the other hand, for the granulate G to remain inside the container 2, the passage openings 41 have a diameter which is smaller than the mean diameter of the granulate. For example, the separation element 4 may be a sieve having a multiplicity of round openings with a diameter of 0.6 mm, so that granulate G with a mean granulate diameter of about 1 mm is retained inside the interior space 215 of the container. The expression mean granulate diameter is used here to denote the mean grain size, as determined by a screen analysis according to DIN EN 933-1:2012-03. The container opening 24, through which the liquid F can be admitted into the container 2, and which is closed with the removable closure 5 before using the mixing and discharge system 1, is situated distally to the separation element 4.

Represented in FIG. 4 is a separation element 4′ according to a second embodiment, which, unlike the integrally formed separation element in FIGS. 1 to 3, is formed separately from the container 2 and can be introduced subsequently into the interior space 215 of the container.

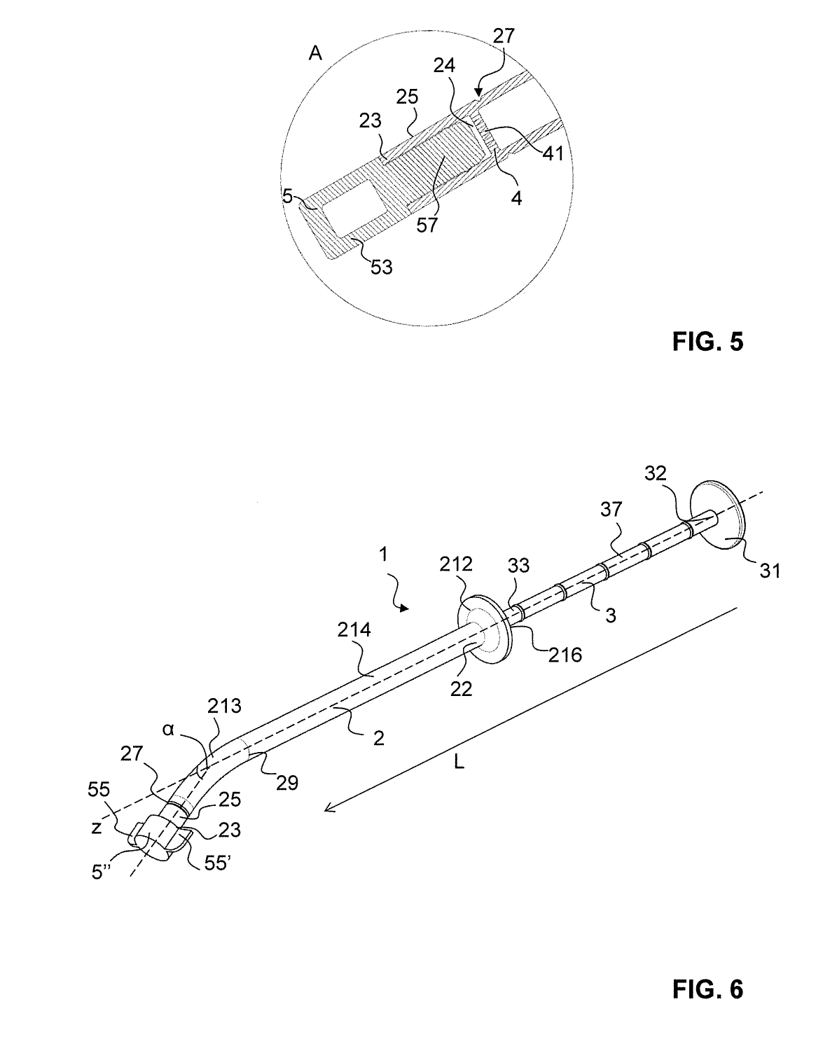

As can be appreciated from FIGS. 1 to 4, the closure 5 has a plug 57 and is pushed together with it into the container opening 24. In the distal direction, the closure 5 protrudes beyond the distal container end 23 and is provided with a grip 53, which can be grasped firmly by a user for the purpose of removing the closure 5.

Whereas the closure 5 in this case projects only partially into the container opening 24 and is arranged in the container 2 at a distance from the separation element 4, however, the closure can also have a plug which extends through the distal end region 25 as far as the separation element 4. A suchlike closure 5′ is depicted in FIG. 5 and has the additional advantage that it prevents fine granules, of which the diameter is smaller than the diameter of the passage openings 41 of the separation element 4, from accumulating in the distal end region 25 between the separation element 4 and the closure plug.

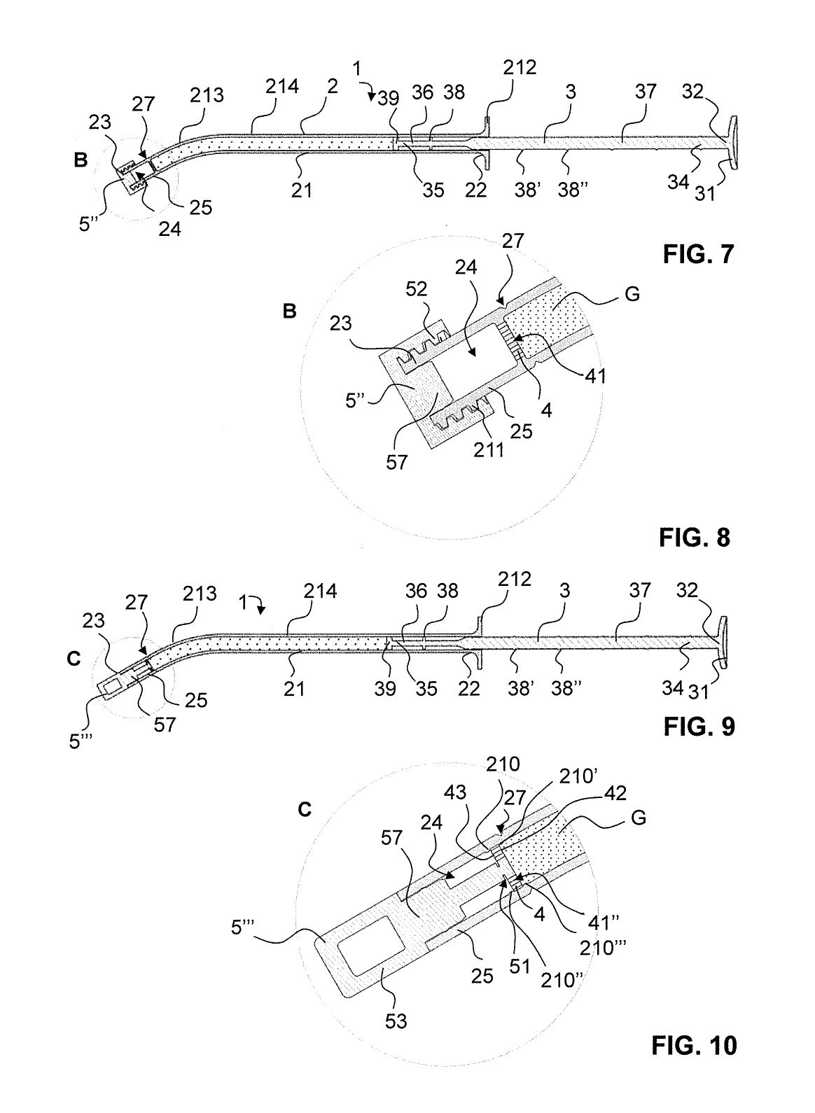

The mixing and discharge apparatus 1 in FIGS. 6 to 8 has a closure according to second embodiment, wherein the closure is configured as a closure cap 5″ and in this case possesses an internal thread 52. The container 2 has a corresponding external thread 211 in the region of the container opening 24, so that the closure cap 5″ can be screwed onto the distal container end 23. It is also conceivable, however, for the closure cap 5″ to be configured without an internal thread, and for the container 2 not to have an external thread, so that the closure cap 5″ is simply pushed onto the distal container end 23. It is conceivable, furthermore, for the closure cap 5″ also to have a plug 57, which extends through the distal end region 25 as far as the separation element 4. The closure cap 5″ in this case is provided with retaining wings 55, 55′ for easier handling.

Various combinations of closures 5′″, 5″″, 5′″″, having a respective separation element 4″, 4′″, 4″″, are represented in FIGS. 9 to 16. In all of these embodiments, the closure 5′″, 5″″, 5′″″ in each case is connected initially to the separation element 4″, 4′″, 4″″, wherein the separation element 4″, 4′″, 4″″ is attached to the container 2 in such a way that the separation element 4″, 4′″, 4″″ remains in the container 2 when the closure 5′″, 5″″, 5′″″ is removed from the container 2. For this purpose, the closure 5′″, 5″″, 5′″″ is connected integrally to the separation element 4″, 4′″, 4″″ via a predefined breaking point 51.

A suchlike embodiment permits simplified production of the mixing and discharge system 1, since the joint production of the separation element 4″, 4′″, 4″″ and the closure 5′″, 5″″, 5′″″ is simpler, if only for production engineering reasons, compared with production of the container 2 with an integrated separation element 4. In addition, assembly of the separation element 4″, 4′″, 4″″ in the container 2 is also made easier by a separation element 4″, 4′″, 4″″ connected to the closure 5′″, 5″″, 5′″″, since the separation element 4″, 4′″, 4″″ can be introduced into the container 2 by means of the closure 5′″, 5″″, 5′″″. In addition, separate production of the separation element 4″, 4′″, 4″″ also permits adaptations to the mixing and discharge apparatus 1 to different types of granulate for the same embodiment of container in each case, since the separation element 4″, 4′″, 4″″ can be provided with different passage openings 41′, 41″, 41′″, 41″″, which correspond to the grain size of the respective granulate.

In order to prevent displacement of the separately formed separation element 4″ in the interior space 215 of the container in the proximal direction and also in the distal direction, the container wall 21 according to FIG. 10 comprises inwardly projecting retaining means in the form of protrusions 210, 210′, 210″, 210′″, which are arranged both proximally and distally to the separation element 4″ and thereby secure the separation element 4″ in the container 2. The protrusions 210, 210′, 210″, 210′″ in this case are configured integrally with the container wall 21 and extend from the container wall 21 in the direction of the interior space 215 of the container at least partially along the proximal side 42 and the distal side 43 of the separation element 4″, wherein the protrusions 210, 210′, 210″, 210′″ directly adjoin the proximal or distal side 42, 43 of the separation element 4″.

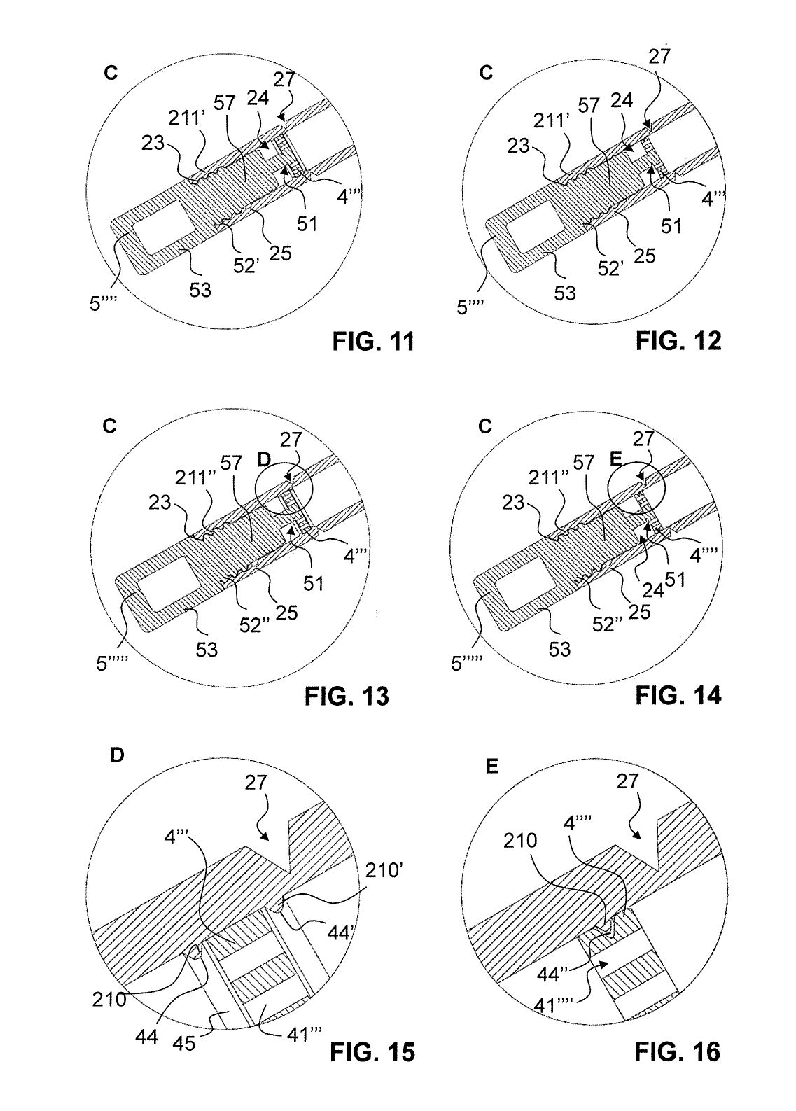

As an alternative or in addition, however, the separation element 4′″, 4″″ may possess lateral recesses 44, 44′, 44″, in which the protrusions 210, 210′ engage and thereby secure the separation element 4′″, 4″″ against rotation inside the container 2. As apparent in FIGS. 11, 13 and 15 or 12, 14 and 16, the separation element 4′″ can be secured in the container 2, for example bilaterally, with respectively two protrusions 210, 210′, which protrude into corresponding lateral recesses 44, 44′ in an edge of a separation element (see FIG. 15), or bilaterally with respectively one protrusion 210, which protrudes into a lateral, centrally configured recess 44″ in the separation element 4″″ (see FIG. 16).

The closure 5″″, 5′″″ is also shown in FIGS. 11 to 16 to be secured to the container in each case by means of a screwed connection. As represented in FIGS. 11 and 12 and FIGS. 13 and 14 respectively, the container wall 21 can have a round thread 211′ or a buttress thread 211″ internally in the distal end region 25, which engages with a corresponding external thread 52′, 52″ on the closure plug 5″″, 5′″″. The provision of a suchlike threaded connection brings the advantage that the closure 5″″, 5′″″ together with the separation element 4′″, 4″″ can be pushed into the container 2 through the container opening 24 in the proximal direction in the course of assembly. Subsequent removal of the closure 5″″, 5′″″ by pulling on the closure in the distal direction is prevented, however, by the engagement of the thread between the internal thread 211′, 211″ of the container 2 and the external thread 52′ 52″ of the closure 5″″, 5′″″. Instead, the closure 5″″, 5′″″ can be removed from the container 2 simply by means of a screwing movement. The screwing movement causes both tensile forces and torsional forces to act upon the predefined breaking point 51 between the separation element 4′″, 4″″ and the closure 5″″, 5′″″, so that the predefined breaking point 51 breaks with the expenditure of less force than would be the case for purely axial withdrawal.

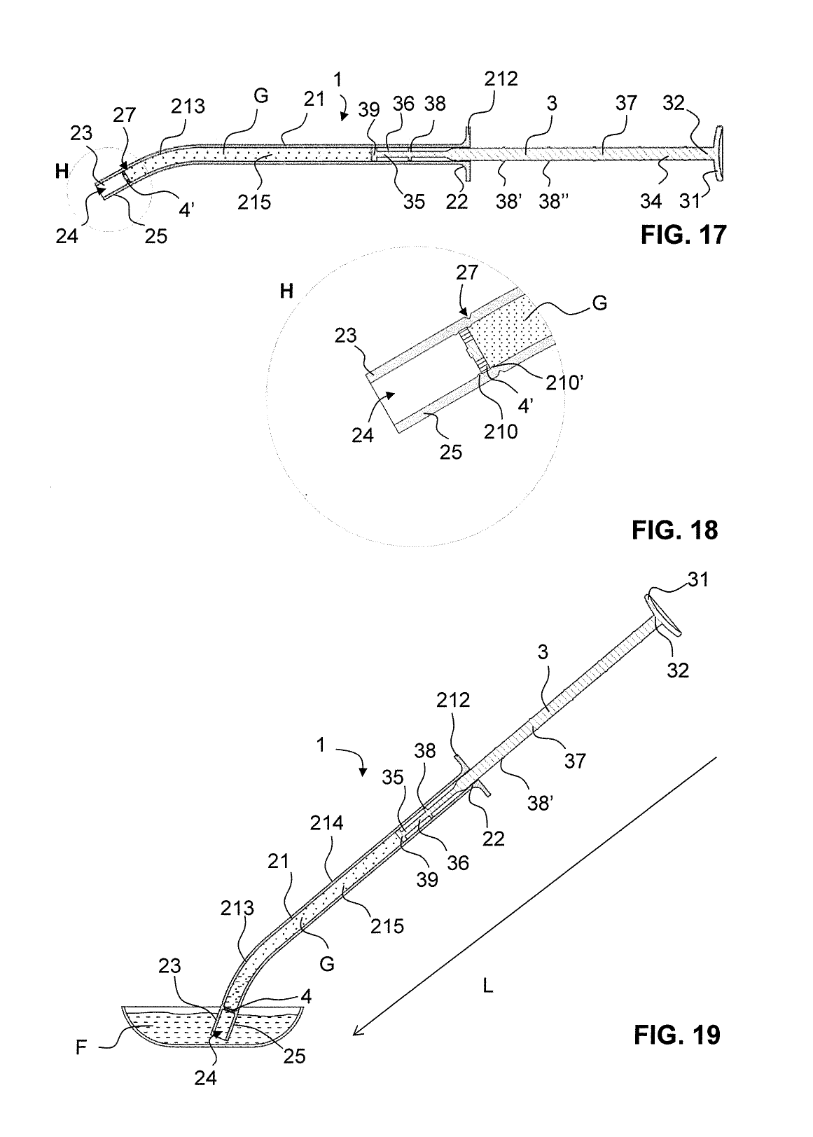

Once the closure 5, 5′, 5″, 5′″, 5″″, 5′″″ has been removed from the mixing and discharge apparatus 1, the container opening 24 is exposed. This state is represented in FIGS. 17 and 18.

The granulate G in the interior space 215 of the container is now impregnated with liquid F. One possibility for impregnating the granulate G involves, as depicted in FIG. 19, the liquid F being sucked up into the interior space 215 of the container. For this purpose, the mixing and discharge system 1 with its exposed container opening 24 is immersed in a reservoir of liquid F, and sucking-up of the liquid takes place by the feed element 3 being drawn back in the container 2 against the discharge direction L. In the process, the impregnation liquid passes through the passage openings 41, 41′, 41″, 41′″, 41″″ of the separation element 4, 4′, 4″, 4′″, 4″″ into the interior space 215 of the container.

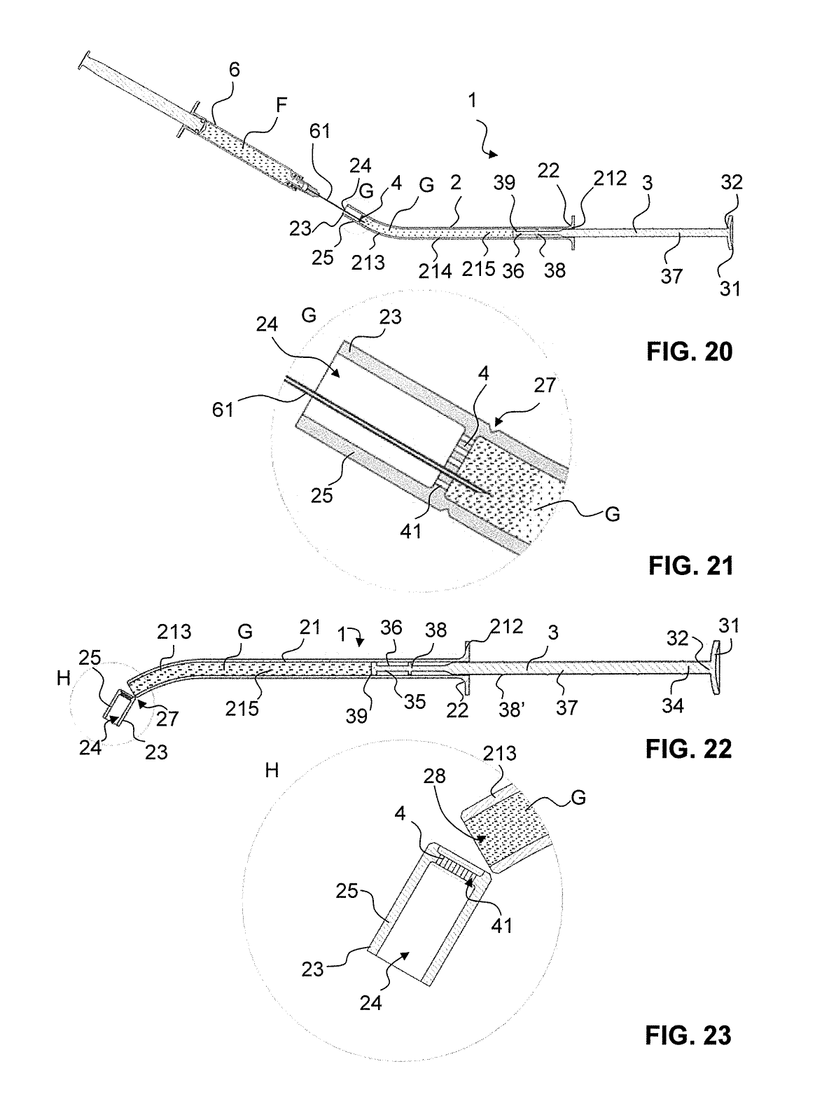

A further possibility for impregnating the granulate G is represented in FIG. 20, wherein liquid F is injected into the interior space 215 of the container through the container opening 24 and through the passage openings 41, 41′, 41″, 41′″, 41″″ of the separation element 4, 4′, 4″, 4′″, 4″″. A moistening syringe 6 can be used for this purpose, of which the syringe needle 61 possesses an external diameter which is the same size or smaller than the diameter of the passage openings 41, 41′, 41″, 41′″, 41″″ of the separation element 4, 4′, 4″, 41′″, 4″″. As a result, the syringe needle 61 can be introduced directly through the passage openings 41, 41′, 41″ without first penetrating the separation element by the expenditure of force.

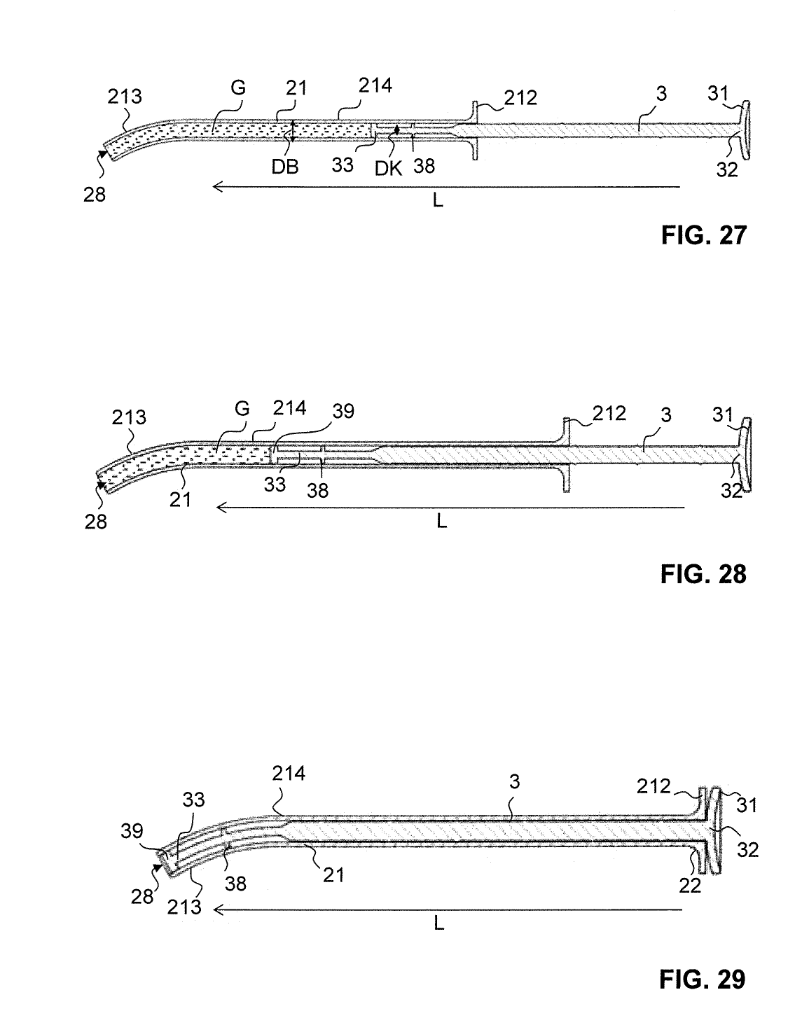

Once the granulate G has been impregnated with the liquid, the finished bone graft material can be discharged from the mixing and discharge apparatus 1. However, the separation element 4, 4′, 4″, 4′″, 4″″ must be removed from the mixing and discharge apparatus 1 for this purpose. As already mentioned, the container 2 has a predefined breaking point 27 proximal to the separation element 4, 4′, 4″, 4′″, 4″″, so that the distal end region 25 situated between the predefined breaking point 27 and the distal end 23 of the container can be broken off from the container 2 together with the separation element 4, 4′, 4″, 4′″, 4″″. This is illustrated in FIGS. 22 and 23. As is apparent from FIGS. 27 to 29, a discharge opening 28 for discharging the mixture is provided in the container 2 in this way. As is further apparent from FIGS. 22 and 23, the predefined breaking point 27 is present in the angled section 213 of the container 2, so that the discharge opening 28, once the distal end region 25 has been broken off, is likewise present in the angled section 213 of the container 2.

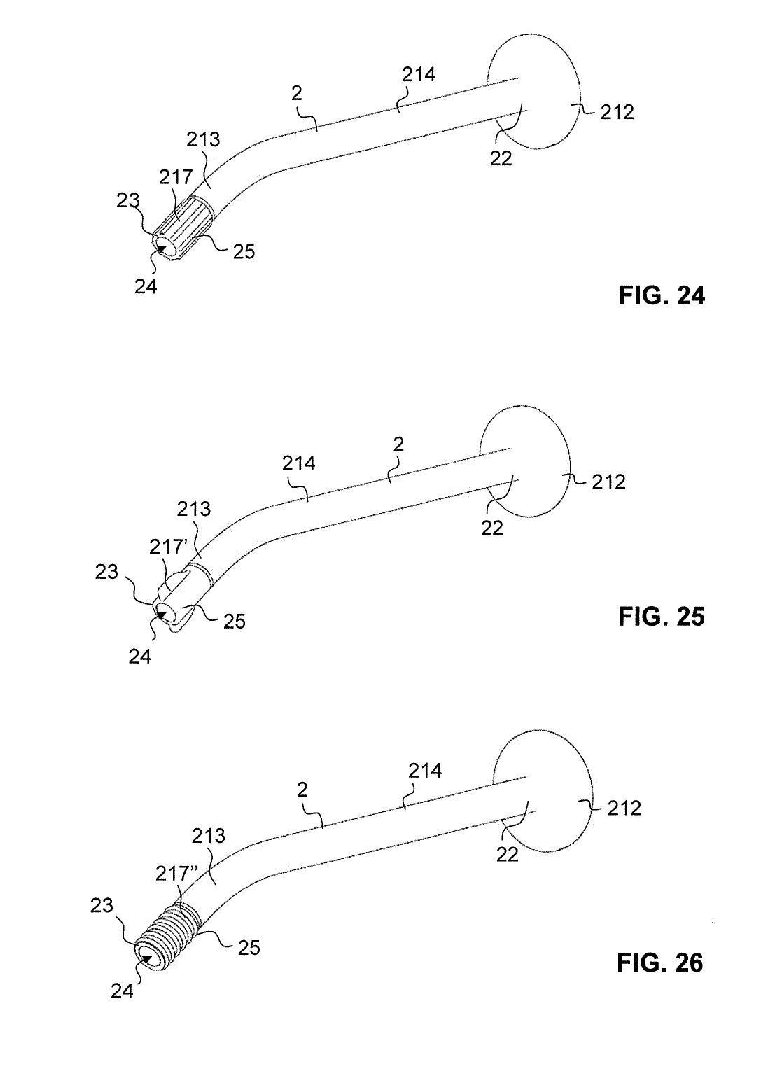

As can be seen in FIGS. 24 to 26, the distal end region 25 of the container 2 can have an external aid to breaking, for example in the form of longitudinally oriented ribs 217, retaining wings 217′ or transversely running grooves 217″, which provide the user with an improved grip and in so doing facilitate the breaking-off of the distal end region 25. As an alternative thereto, however, the closure cap can also serve as an aid to breaking. If, for example, the closure cap 5′ in FIG. 6 is positioned once more on the distal end 23 of the container following impregnation of the granulate G, these retaining wings 55, 55′ can be gripped by a user, and the distal end region 25 of the container 2 together with the closure cap 5″ can be broken off. Of course, all of the closures disclosed herein can be positioned once more on the distal end 23 of the container following impregnation of the granulate and can be gripped for the purpose of breaking-off the distal end region 23.

The container 2 is configured in the present illustrative embodiments as a cylindrical tube, which defines a constant interior diameter DB. In order to reduce the friction between the container wall and the piston rod, the piston rod 37 of the feed element 3 in a proximal region 34 has an exterior diameter which is a little smaller than the interior diameter DB of the container 2. Nevertheless, in order to guarantee good guidance of the piston rod 37 in the interior space 215 of the container, the piston rod 37 has radially outwardly projecting, circumferential, annular guide beads 38, 38′, 38″, 38′″, which bear against the container wall 21. In order for the piston rod 37 also to be able to follow the angled section 213 of the container 2, the piston rod 37 has a region of further reduced diameter 36 in a distal region 35. The reduced diameter DK of the piston rod 37 in this case is smaller than the diameter in the proximal region 34 of the piston rod 37, which imparts good flexibility to the piston rod 37 in the distal region of the container 2.

FIG. 27 depicts the mixing and discharge apparatus 1 in an initial state, at the start of the discharge of the mixture. The distal end region 25 of the container 2 has been broken off together with the separation element 4, 4′, 4″, 4′″, 4″″, and the piston rod 37 has been drawn back in the proximal direction and extends parallel to the central longitudinal axis Z.

In FIG. 28, the mixing and discharge apparatus 1 is represented in an intermediate state, wherein the piston 39 has been advanced partially into the container 2 in the distal discharge direction L and has discharged a part of the mixture. If the piston 39 is now advanced more in the distal direction, both the piston 39 and the distal region 35 of the piston rod 37 follow the angled section 213 of the container 2, wherein this flexibility of the piston rod 37 is assured in particular by the region of reduced diameter 36 of the piston rod 37.

FIG. 29 depicts the end state, in which the piston 39 has been advanced in the distal direction as far as the discharge opening 28 of the container 2 and the piston rod 27 has been received completely in the interior space 215 of the container. The longitudinal dimension of the piston 39 and the region of reduced diameter 36 in this case corresponds directly to the longitudinal dimension of the angled section 213 of the container 2. As can be seen in FIG. 28, the piston 39 as well as the guide bead 38 in this case bear against the container wall 21 of the container 2 in a sealing manner.

### LIST OF REFERENCE SIGNS

1 mixing and discharge system
2 container
21 container wall
22 proximal end of the container
23 distal end of the container
24 container opening
25 distal region
27 predefined breaking point
28 discharge opening
29 region of curvature
210-210′″ retaining means
211-211′″ thread
212 retaining plate
213 angled section
214 straight region
215 interior space of the container
216 inlet opening
217-217″ aid to breaking
3 feed element
31 pressure plate
32 proximal end
33 distal end
34 proximal region
35 distal region
36 tapering
37 piston rod
38 guide bead
39 piston
4-4″″ separation element
41-41″″ passage openings
42 proximal side
43 distal side
44-44″ lateral recess
45 edge of separation element
5-5″″ closure
51 predefined breaking point
52-52″ thread
53 grip
54 proximal extension
55-55′ retaining wings
57 plug
6 moistening syringe
61 needle
L discharge direction
Z central longitudinal axis
α angle of curvature
DB container diameter
DK piston diameter
G granulate
F liquid

1. A mixing and discharge system for impregnating granulate with a liquid and for discharging a resulting mixture, comprising:
a container for holding the granulate, having a circumferential container wall, a proximal container end and a distal container end, wherein the distal container end forms a container opening for introducing the liquid into the container;
a feed element displaceably mounted in the container for discharging the mixture in a distal discharge direction; and
a separation element arranged in a distal end region of the container, which element is designed to retain granulate but to allow liquid to pass through;
wherein the container has a predefined breaking point proximal to the separation element so that the distal end region situated between the predefined breaking point and the distal container end can be broken off from the container together with the separation element, thereby creating a discharge opening on the container for discharging the mixture.

2. The mixing and discharge system as claimed in claim 1, wherein the separation element is configured in one piece with the container wall.

3. The mixing and discharge system as claimed in claim 1, wherein the separation element is formed separately from the container.

4. The mixing and discharge system as claimed in claim 3, wherein the container wall has at least one retaining means, which secures the separation element in the container at least in relation to the distal application direction.

5. The mixing and discharge system as claimed in claim 4, wherein at least one of the retaining means secures the separation element in relation to rotations in the container.

6. The mixing and discharge system as claimed in claim 1, further having a removable closure in order to close the container opening.

7. The mixing and discharge system as claimed in claim 6, wherein the closure, has a plug, which extends against the discharge direction into the container through the container opening as far as the separation element.

8. The mixing and discharge system as claimed in claim 6,
wherein the closure is connected to the separation element, and
wherein the separation element is secured to the container in such a way that the separation element remains inside the container if the closure is removed from the container.

9. The mixing and discharge system as claimed in claim 8, wherein the closure is formed in one piece with the separation element.

10. The mixing and discharge system as claimed in claim 9, wherein the closure is connected to the separation element by means of a predefined breaking point.

11. The mixing and discharge system as claimed in claim 1,
wherein the container has a curvature and forms, distally from the curvature, an angled section which extends outwards from the central longitudinal axis in relation to a central longitudinal axis of the container,
wherein the distal end region is arranged in the angled section.

12. The mixing and discharge system as claimed in claim 11,
wherein the container defines an internal diameter,
wherein the feed element has a piston, which is connected to a piston rod, and wherein the piston rod has a region of reduced external diameter in a distal region,
wherein the reduced external diameter is smaller than the internal diameter, so that the piston rod is at a distance from the container wall in the region of the reduced external diameter, and
wherein the piston rod in the region of reduced external diameter has at least one radially outward-projecting guide bead, which bears against the container wall.

13. The mixing and discharge system as claimed in claim 1,
wherein the container is pre-filled with granulate having a medium-sized granulate diameter, and
wherein the separation element has passage openings, of which the diameter is smaller than the medium-sized granulate diameter.

14. A method for impregnating a granulate with a liquid, comprising:
providing a mixing and discharge system pre-filled with granulate as claimed in claim 1;
impregnating the granulate with a liquid;
breaking off the distal end region together with the separation element at the predefined breaking point, in order to create the discharge opening for discharging the mixture on the container; and
displacing the piston rod in the distal discharge direction in order to discharge the mixture from the discharge opening.

15. The method as claimed in claim 14, wherein impregnation of the granulate takes place by absorption of the liquid by causing the feed element to move against the discharge direction.

16. The method as claimed in claim 14, wherein impregnation of the granulate takes place by injection of the liquid through the container opening and through the separation element.

17. The method as claimed in claim 16, wherein injection of the liquid takes place by the introduction of a needle into the container through preformed openings in the separation element.

18. The mixing and discharge system as claimed in claim 11, wherein the angled section extends outwards from the central longitudinal axis at an angle of curvature of between 20° and 50°.
